(19) United States
(12) Patent Application Publication
(10) Pub. No.: US 2008/0064924 A1
(43) Pub. Date: Mar. 13, 2008

(54) Bronchoscope with wireless image transmission

(76) Inventor: Tien-Sheng Chen, Taipei (TW)

Correspondence Address: Andrews & Kurth, L.L.P., Suite 1100, 1350 I Street N.W., Washington, DC, 20005, US

(21) Appl. No.: 11/604,346

(22) Filed: Nov. 27, 2006

(30) Foreign Application Priority Data
Sep. 13, 2006 (TW) 095216313

(51) Int. Cl. A61B 1/04; A61B 1/06

(52) U.S. Cl. 600/109; 600/179

(57) ABSTRACT

A bronchoscope with wireless image transmission has a power device, a wireless transmitter and a flexible snake camera. The snake camera electrically connects to the power device and has a first end and a second end. The first end of the snake camera connects to the transmitter. The second end of the snake camera is a camera end. Hence, the picture can be seen on a screen to smooth operation.

### BACKGROUND OF THE INVENTION

1. Field of the Invention

The present invention relates to a bronchoscope, in particular, to a bronchoscope with wireless image transmission.

2. Description of the Related Art

A plastic endotracheal tube should be inserted into the patient's trachea to maintain breathing of the patient. During the insertion of the endotracheal tube into the trachea difficulty is encountered in about 20% patients due to their short chins or stiff necks. So, a first conventional bronchoscope is used as an auxiliary tool.

The first conventional bronchoscope has a flexible tip with lens to take the image, optical fibers mounted therein, a shaft and a driving member. When the first conventional bronchoscope is inserted into the patient's trachea, the doctor can look at the tracheal condition and the endotracheal tube can be mounted around it and then pushed into the trachea to supply oxygen. The tip with lens can be folded and driven by the shaft for easy operation. An eyepiece, at the other end, connects to the tip with lens to receive the image from it through optical fibers.

However, the quality of the image transmitted by the bronchoscope is limited by the amount of the optical fibers. Furthermore, because the user uses the shaft to adjust the bronchoscope, it is easily shaken during operation.

Therefore, the invention provides a bronchoscope for scrutiny with wireless image transmission to mitigate or obviate the aforementioned problems.

### SUMMARY OF THE INVENTION

The main objective of the present invention is to provide a bronchoscope for scrutiny with wireless image transmission that is easy to operate with clear image.

Other objectives, advantages and novel features of the invention will become more apparent from the following detailed descriptions when taken in conjunction with the accompanying drawings.

### BRIEF DESCRIPTION OF THE DRAWINGS

FIG. 1 is a side view in partial section of a bronchoscope with wireless image transmission in accordance with the present invention;

FIG. 2 is a side view in partial section of the bronchoscope with wireless image transmission in FIG. 1;

FIG. 3 is a side view of a display of the bronchoscope with wireless image transmission in FIG. 1; and

FIG. 4 is a block diagram of the bronchoscope with wireless image transmission in FIG. 1.

### DETAILED DESCRIPTION OF THE PREFERRED EMBODIMENT

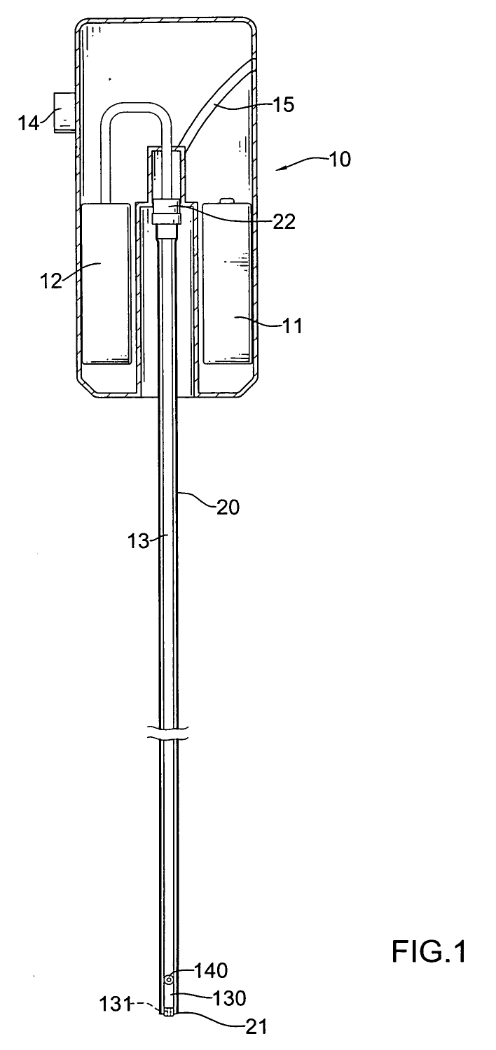

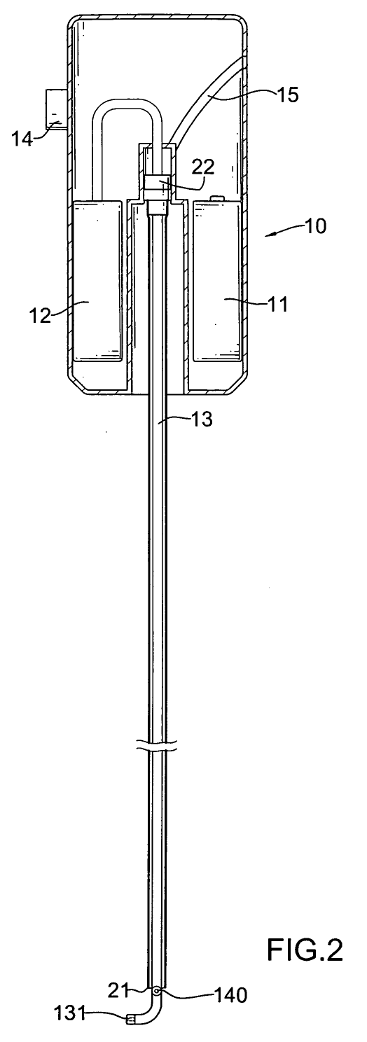

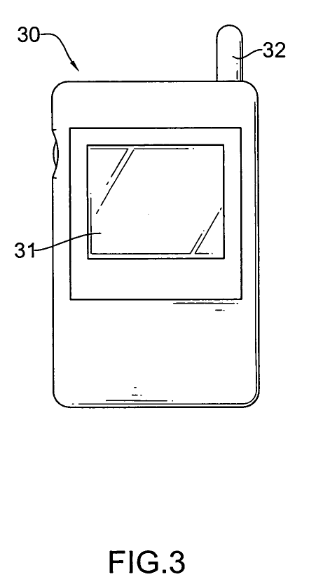

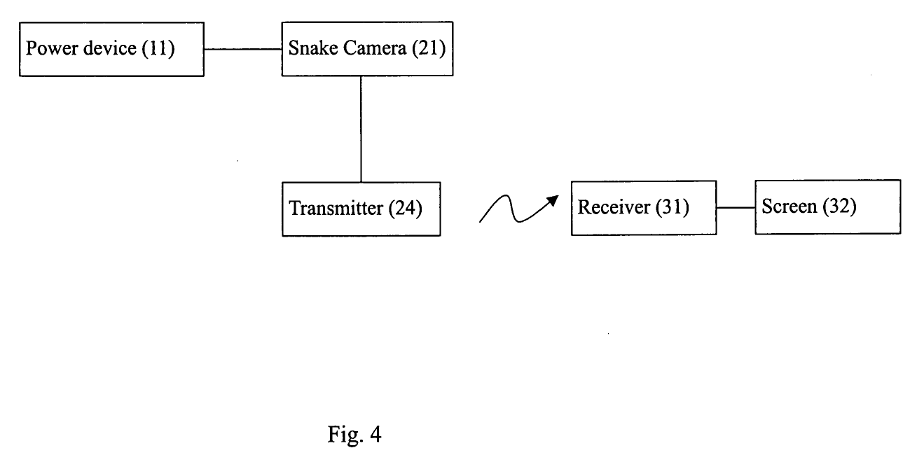

With reference to FIGS. 1 to 4, a bronchoscope with wireless image transmission in accordance with the present invention has a power device (11), a wireless transmitter (12), a snake camera (13), a button (14), a vent passage (15) and a display (30).

The power device (11) is a battery for power supply.

The snake camera (13) is electrically connected to the power device (11), is flexible and has a camera end (130). The snake camera (13) may have CCD-based (Charge Coupled Device) camera or a CMOS-based (Complementary Metal-Oxide Semiconductor) camera. A first end of the snake camera (13) connects to the transmitter (12) and the second end of the snake camera (13) is the camera end (130) having an LED light source (131) mounted in the camera end (130). An endotracheal tube (20) is mounted around the bronchoscope (10) and has a vent (21) defined in a first end of the endotracheal tube (20) and a conjuncture (22) mounted in a second end of the endotracheal tube (20).

The wireless transmitter (12) electrically connects the snake camera (13) to transmit the image taken by the camera end (130).

The button (14) connects to the camera end (130) of the snake camera (13) and connects to a driving member (140) so that the camera end (130) can be adjusted when the button (14) is pressed. The driving member (140) can be a transmission that can drive the camera end (130) to move. The vent passage (15) is defined in the bronchoscope (10) for auxiliary ventilation.

The display (30) has a screen (31) and a receiver (32). The receiver (32) can convert the signal to the image to show on the screen (31).

When the bronchoscope (10) is inserted into the patient's trachea, the camera end (130) can take image of the tracheal condition and the image can be transmitted by the transmitter (12) and converted by the receiver (32) to show on the screen (31).

When a patient needs to be intubated, the bronchoscope is used as an auxiliary instrument. The camera end (130) of the snake camera (13) is adjusted by pressing the button (14) to locate the trachea of the patient. Pushing the endotracheal tube (20) to slide along the snake camera (13) and the vent (21) of the endotracheal tube (20) will slide into the trachea of the patient. The conjuncture (22) of the endotracheal tube (20) connects to a feeding-oxygen device to deliver the oxygen through the vent (21) into the trachea of the patient.

The wireless transmission between the transmitter (12) and the display (30) simplifies the structure of the present invention. Furthermore, the camera end (130) of the snake camera (13) is adjusted by the button (14) to avoid shaking.

It is to be understood, however, that even though numerous characteristics and advantages of the present invention have been set forth in the foregoing description, together with details of the structure and function of the invention, the disclosure is illustrative only. Changes may be made in details, especially in matters of shape, size, and arrangement of parts within the principles of the invention to the full extent indicated by the broad general meaning of the terms in which the appended claims are expressed.

What is claimed is:

1. A bronchoscope for scrutiny with wireless image transmission comprising:
a power device mounted in the bronchoscope;
a wireless transmitter mounted in the bronchoscope; and
a flexible snake camera electrically connected to the power device and having
a first end connecting to the wireless transmitter; and
a second end being a camera end.

2. The bronchoscope with wireless image transmission as claimed in claim 1 further has a button connecting to the camera end of the snake camera and connecting to a driving member.

3. The bronchoscope with wireless image transmission as claimed in claim 1, wherein the camera end has an LED.

4. The bronchoscope with wireless image transmission as claimed in claim 3, wherein the power device is a battery.
